(19) United States
(12) Patent Application Publication
(10) Pub. No.: US 2024/0077480 A1
(43) Pub. Date: Mar. 7, 2024

(54) LATERAL FLOW ASSAY FOR DETECTING PATHOGENS IN MILK FROM MASTITIC COWS

(71) Applicant: Zoetis Services LLC, Parsippany, NJ (US)

(72) Inventors: Sridhar Velineni, Kalamazoo, MI (US); Amanda Faye Kenworthy, Vicksburg, MI (US)

(21) Appl. No.: 18/548,987

(22) PCT Filed: Mar. 7, 2022
(86) PCT No.: PCT/US2022/019070

§ 371 Date: Sep. 5, 2023

Related U.S. Application Data
(60) Provisional application No. 63/157,179, filed on Mar. 5, 2021.

(51) Int. Cl. G01N 33/569; G01N 33/543

(52) U.S. Cl. CPC G01N 33/56944; G01N 33/54388; G01N 2333/31; G01N 2333/315; G01N 2469/10; G01N 2800/365

(57) ABSTRACT

The present invention provides a lateral flow device for detecting lipoteichoic acid (LT A) as a Gram-positive bacteria identifier in a milk sample of an animal. The device comprises: a) a strip formed of a material enabling capillary flow of fluid along a portion of the strip; b) a sample pad located proximal to one end of the strip for receiving the milk sample, c) a conjugate pad located in the strip so that in operation the sample flows under capillary action through the strip from the sample pad to the conjugate pad and mobilizes a conjugate contained in the conjugate pad, the conjugate comprising an anti-LTA antibody that has been conjugated to a detection agent, d) a test line Test Die comprising an anti-LTA antibody immobilized within the strip along a band located substantially perpendicular to the direction of the sample flow along the strip so that when a formed complex comprised of the mobilized anti-LTA antibody conjugate and the LT A in the sample contacts the immobilized anti-LTA antibody in the test line the presence of LT A in the sample is indicated by a visible color change.

### FIELD OF THE INVENTION

The present invention relates to an enrichment-based lateral flow (LF) test for the direct detection of target Gram-positive bacteria in milk samples collected from individual quarters of mastitic cows.

### BACKGROUND OF THE INVENTION

Cows are normally milked at least twice a day. Most dairies have enough machines to milk more than twenty cows at one time. Milking machines mimic the action of a young calf by creating a pulsating vacuum around the teat, which causes the milk to be released from the udder.

Milk is usually stored on the farm at cold temperatures in milk storage vats or silos for no more than 48 hours. After milk has been collected, storage vats and stainless steel pipes are thoroughly cleaned before the farmer milks again.

Milk is collected from the farm every 24 or 48 hours by tankers that have special stainless steel bodies which are heavily insulated to keep the milk cold during transportation to the processing factory. Milk tanker drivers are accredited milk graders, qualified to evaluate the milk prior to collection. The tanker driver grades and, if necessary, reject milk based on temperature, sight, and smell. A representative sample is collected from each farm pickup prior to being pumped onto the tanker. After collection, milk is transported to factory sites and stored in refrigerated silos before processing.

Samples of milk are taken from farm vats prior to collection and from the bulk milk tanker upon arrival at the factory. Samples from the bulk milk tanker are tested for antibiotics and temperature before the milk enters the factory processing area. Farm milk samples are tested for milkfat, protein, bulk milk cell count and bacteria count. If milk does not meet quality standards it is rejected. Most farmers are paid on the quality and composition of their milk.

Mastitis is the inflammation of the mammary gland and udder tissue due to microbial infection or physical trauma. It continues to be the most frequent and costliest disease of dairy cattle globally. Financial losses due to mastitis occur for both sub-clinical and clinical disease. Clinical mastitis is readily apparent and easily detected by abnormalities in milk or the udder or the occurrence of secondary clinical signs. Current diagnosis of sub-clinical mastitis is made based on the outcome of indirect tests such as somatic cell count (SCC), the California Mastitis Test (CMT) or milk conductivity as an indirect indicator of infection. Typically, when mastitis is diagnosed, the cow is milked out and the infected quarter is treated with an antibiotic by intramammary infusion.

Early detection of mastitis improves treatment outcome and the ability to detect Gram-positive bacteria drives treatment decisions. All currently licensed intramammary infusion products are labelled to treat Gram-positive mastitis pathogens (e.g., staphylococci and streptococci). Fewer antimicrobials are labelled to treat a Gram-negative mastitis pathogen (e.g., Escherichia coli). Regulatory and environmental pressures continue toward a trend to reduce the use of broad-spectrum antimicrobials and/or blanket therapy in the management of bovine mastitis. As such, it become increasingly critical to accurately and quickly diagnose etiological pathogen(s) responsible for mastitis to facilitate the targeted use of narrow-spectrum antimicrobials and ensure treatment success and positive outcomes. It would be desirable to focus diagnostic efforts on early diagnosis of mastitis (e.g., based on clinical signs, dairy records, SCC, etc.), followed by determination of the etiological pathogen to initiate timely and appropriate antibiotic treatment.

Currently, diagnosis of mastitis typically relies on (1) SCC as an indirect indicator of infection and (2) in vitro milk culture, a laboratory-based pathogen identification method that typically requires milk samples to be shipped with a typical turnaround time of days to weeks. Both methods do not satisfy the need of dairy farmers or veterinarians for the early detection and identification of mastitis pathogens. Therefore, it would be desirable to provide for point-of-care tests that detect mastitis pathogens in milk to guide antimicrobial selection. In particular, a Gram-positive identifier diagnostic would meet a customer need and complement treatment options on the market, thereby providing a more comprehensive customer solution tailored to the individual animal. It would be desirable if the point-of-care test was performed after the first milking, but before the next milking in order to help ensure the quality and composition of the milk collected from the farm and reduce financial losses while at the same time improving treatment outcome.

### SUMMARY OF THE INVENTION

Lipoteichoic acid (LTA) is the major proinflammatory structure present within the cell wall layer of almost all Gram-positive bacteria. It plays an important role in the initiation and progression of bacterial infection, inflammation, and septic shock. The present invention provides a lateral flow device for detecting LTA expressed on the surface of Gram-positive bacteria as a mastitis Gram-positive bacteria identifier in a milk sample of an animal. The device comprises: a) a strip formed of a material enabling capillary flow of fluid along a portion of the strip; b) a sample pad located proximal to one end of the strip for receiving the milk sample, c) a conjugate pad located in the strip so that in operation the sample flows under capillary action through the strip from the sample pad to the conjugate pad and mobilizes a conjugate contained in the conjugate pad, the conjugate comprising an anti-LTA antibody that has been conjugated to a detection agent, d) a test line comprising an anti-LTA antibody immobilized within the strip along a band located substantially perpendicular to the direction of the sample flow along the strip so that when a formed complex comprising the mobilized anti-LTA antibody conjugate and the LTA in the sample contacts the immobilized anti-LTA antibody in the test line, the presence of LTA in the sample is indicated by a visible color change. In one embodiment, the strip is formed of nitrocellulose.

In one embodiment, the milk sample has been enriched for bacterial cells. In another embodiment, the milk sample is from a mastitic quarter of a cow.

In a further embodiment, the milk sample is from an animal selected from the group consisting of a canine, a feline, equine, caprine, ovine, or a bovine animal. In a specific embodiment, the milk sample is from a bovine animal.

In one embodiment, the device further includes a wicking pad for receiving and retaining sample after passing through the test line and an optional control line.

In another embodiment, the anti-LTA antibody in the conjugate and in the test line is a monoclonal antibody. In one embodiment, the detection agent conjugated to the anti-LTA antibody is selected from the following: metallic nanoparticles or nanoshells, non-metallic nanoparticles or nanoshells, enzymes, and fluorescent molecules. In a specific embodiment, the detection agent comprises nanoparticles or nanoshells of metallic gold.

In one embodiment, the lateral flow device is a dipstick. In another embodiment, the sample pad portion of the dipstick is immersible in the milk sample. In one embodiment, the sample pad portion of the dipstick is immersible in a milk sample that has been enriched for bacterial cells. In a further embodiment of the device, the strip is housed within a cassette.

In one embodiment, the sample pad portion of the device comprises a filter membrane for removing one or more components from the sample. In a specific embodiment, the one or more components removed from the sample by the filter membrane of the sample pad are cells, cellular material, fats, or particulate matter.

In one embodiment, the device further includes a control line located substantially perpendicular to the direction of flow of the sample along the strip. In another embodiment, the conjugate pad portion of the device further includes an antibody not specific to Gram-positive bacteria, which is conjugated to a detection agent to form a second antibody conjugate so that in operation the sample flows from the sample pad to the conjugate pad and mobilizes the second antibody conjugate which passes over the test line without reactivity and crosses the control line.

In one embodiment, deposited at the control line of the device is an antibody capable of binding to the mobilized second antibody conjugate as it crosses the control line, said binding at the control line being indicated by a visible color change. In another embodiment, the antibody in the second antibody conjugate is from animal species other than the species from which the milk sample is derived.

In one embodiment, the detection agent conjugated to the antibody in the second antibody conjugate is selected from the following: metallic nanoparticles or nanoshells, non-metallic nanoparticles or nanoshells, enzymes, and fluorescent molecules. In a specific embodiment, the detection agent conjugated to the antibody in the second antibody conjugate comprises nanoparticles or nanoshells of metallic gold.

The present invention further provides a method for detecting lipoteichoic acid (LTA) as a mastitis Gram-positive bacteria identifier in a milk sample from an animal comprising using a device as described according to any of the embodiments described above. In one desired embodiment, the milk sample has been enriched for bacterial cells.

The present invention further provides a method for detecting lipoteichoic acid (LTA) as a mastitis Gram-positive bacteria identifier. The method includes contacting a milk sample from an animal with a conjugate comprising anti-LTA antibody that has been conjugated to a detection agent, wherein an antibody-antigen complex is formed between the anti-LTA conjugate and LTA present on the Gram-positive bacteria in the sample; capturing the formed antibody-antigen complex with an anti-LTA antibody; and detecting the captured complex. In a specific embodiment of this method, the milk sample has been enriched for bacterial cells.

In another embodiment of the method, the anti-LTA antibody in the conjugate and used to capture the antibody-antigen complex is a monoclonal antibody.

In a further embodiment of the method, the detection agent conjugated to the anti-LTA antibody is selected from the following: metallic nanoparticles or nanoshells, non-metallic nanoparticles or nanoshells, enzymes, and fluorescent molecules. In a specific embodiment, the detection agent comprises nanoparticles or nanoshells of metallic gold.

In one embodiment, the methods of the present invention are capable of detecting ≥100 CFU/mL of target Gram-positive bacteria.

### BRIEF DESCRIPTION OF THE DRAWINGS

FIG. 1 is a schematic representation of a lateral flow (LF) device of the invention.

FIG. 2 is a schematic representation of one embodiment of a workflow for an enrichment-based LF assay according to the present invention.

FIG. 3 is a schematic representation showing a comparison of a conventional diagnostic approach for identifying the pathogen in a mastitic cow versus the point-of care diagnostic approach of the present invention.

Other objects, aspects, features, and advantages of the present invention will become apparent from the following description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

### Definitions

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

The terms “Lipoteichoic acid” and its abbreviation “LTA” may be used interchangeably herein. LTA is the major proinflammatory structure present within the cell wall layer of almost all Gram-positive bacteria. It plays an important role in the initiation and progression of bacterial infection, inflammation, and septic shock. LTA is a complex glycosyl-phosphate-containing polymer that is linked via a lipid anchor to the membrane in Gram-positive bacteria.

The term “antibody”, also referred to as “immunoglobulin”, is a Y-shaped protein of the immune system that specifically identifies foreign objects or antigens, such as the components of bacteria, yeasts, parasites, and viruses. Each tip of the ‘Y’ of an antibody contains an antigen-binding site that is specific for a particular epitope on an antigen, such as LTA present on the Gram-positive bacteria, allowing these two structures to bind together with precision. The production of a given antibody is increased upon exposure to an antigen (e.g., a microbial or viral antigen) that specifically interacts with that antibody. Hence, the detection of antigen-specific antibodies in a sample from a subject can inform whether that subject is currently exposed to, or has been previously exposed to, a given microbe, such as a virus, bacteria, fungus, or parasite. An “antibody” typically comprises all or a portion of an Fc region, and may also comprise one or more antigen-binding sites, to facilitate detection by an antibody-specific binding agent, such as an antigen or antigenic peptide. The antibodies can be, e.g., of IgG, IgE, IgD, IgM, or IgA type. In one embodiment, an antibody for use in the device and methods herein is a monoclonal antibody (mAb). In one embodiment, the term “antibody” as used herein may comprise all or a portion of the Fc region, or alternatively it may comprise only the antigen-binding portion of an antibody, such as an Fab fragment.

The term “protein” refers to a polymer of amino acid residues and to variants and synthetic and naturally occurring analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers and naturally occurring chemical derivatives thereof.

The term “antigen” means a molecule having distinct surface features or epitopes capable of stimulating a specific immune response. Antibodies (immunoglobulins) are produced by the immune system in response to exposure to antigens. Antigens maybe proteins, carbohydrates or lipids, although only protein antigens are classified as immunogens because carbohydrates and lipids cannot elicit an immune response on their own.

An “antigen-binding site,” or “binding portion” of an antibody, refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The term “nanoparticles” means uniform particles having a size of 1-200 nm.

The term “nanoshells” means nanoparticles that consist of a core and a metallic shell (usually gold).

Lateral Flow Device and Kits

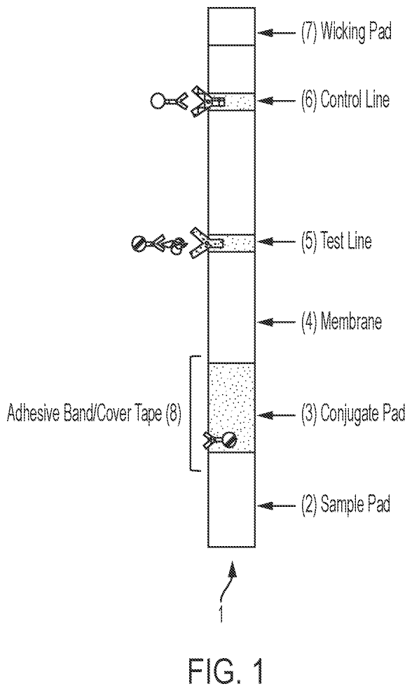

With reference to the figures, FIG. 1 shows a LF device (1) of the invention. The device (1) has a sample pad (2) that makes a sample of bodily fluid from a subject amenable to capillary flow, a conjugate pad (3) including a mobilizable conjugate comprising an anti-LTA antibody that has been conjugated to a detection agent, a membrane (4), and a wicking pad (7) for receiving and holding fluid that has travelled by capillary flow from the sample pad (2) and through the conjugate pad (3) and the membrane (4). A test line (5) is shown which comprises an immobilized anti-LTA antibody. A band (6) is a positive control line. The device (1) can further include an adhesive band or cover tape (8) that connects the sample pad, conjugate pad and nitrocellulose, and a backing (not shown).

When a fluid milk sample is deposited on the sample pad (2), or the sample pad (2) is dipped into the fluid milk sample, fluid travels from the sample pad (2) by capillary flow to the conjugate pad (3) where it comes into contact with an anti-LTA antibody that has been conjugated to a detection agent (not shown). LTA which is abundantly expressed on the surface of Gram-positive bacteria in the sample will react with the anti-LTA conjugate on the conjugate pad (3) to form a complex. The fluid mobilizes the formed complex and transports it to the test line (5). Specifically, the anti-LTA antibody-LTA antigen-detection agent complex formed between any LTA antigen in the sample and the anti-LTA antibody conjugate at conjugate pad (3) migrates across membrane (4) to the test line (5) where the complexed LTA is immobilized by the anti-LTA antibody deposited there. The accumulation of the detection agent on the test line (5) forms a visual signal, i.e., a color change if LTA is present in the sample, indicating a positive result. If LTA is not present in the sample, the conjugate is not immobilized at the test line (5) and continues to migrate to the wicking pad (7). The lack of formation of a visual signal at the test line (5) indicates the sample is negative for LTA meaning that the sample is negative for Gram-positive bacteria since the LTA would be expressed on the surface of Gram-positive bacteria.

In another embodiment of the device (1) in FIG. 1, the conjugate pad (3) further includes an immunoglobulin not specific to Gram-positive bacteria, wherein the immunoglobulin is conjugated to a detection agent (not shown) to form a second antibody conjugate so that in operation the milk sample flows from sample pad (2) to conjugate pad (3) and mobilizes the second antibody conjugate which passes over test line (5) without reactivity and crosses the control line (6). In one embodiment, deposited at the control line (6) is an antibody capable of binding to the mobilized second antibody conjugate as it crosses the control line (6), the binding at the control line (6) being indicated by a visible color change. In one embodiment, the immunoglobulin in the second antibody conjugate is from an animal species other than the species from which the milk sample is derived.

There is a physical overlap and contact between the conjugate pad (3), sample pad (2) and the membrane (4) to allow for LTA-gold conjugate complex formation and proper flow onto the test strip. In one embodiment, the device is a dipstick wherein the conjugate pad, sample pad, and membrane of the dipstick are covered by an adhesive band or cover tape and are housed on a long backing card that provides a 15 mm long handle to the dipstick.

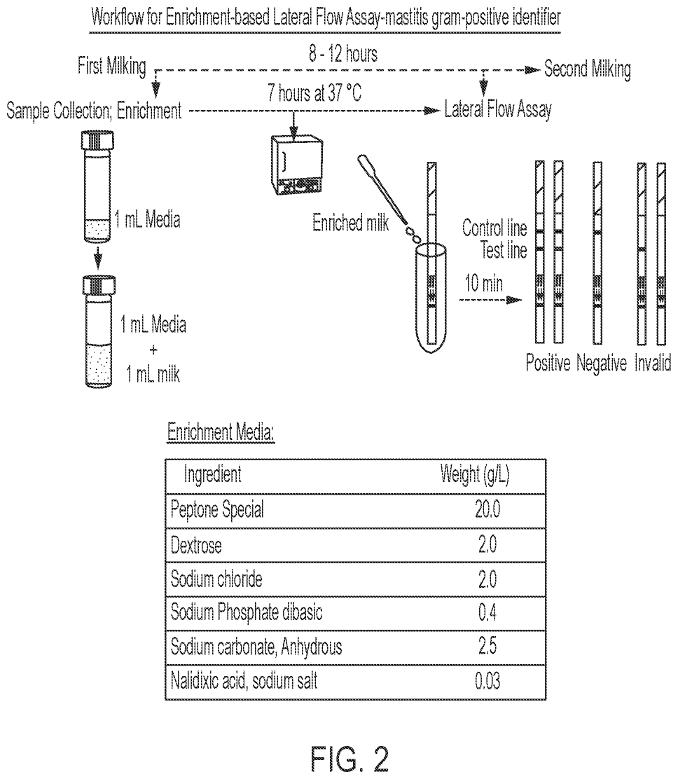

In one embodiment, a sample of raw milk is first enriched according to the enrichment method described below and in FIG. 2 using an enrichment media/broth, a suitable recipe for which is shown in FIG. 2 and disclosed in the examples. It is based on enriching bacterial cells to a detectable level from a milk sample and subsequent detection on a Gram-positive specific LF test. In one embodiment, milk sample from a mastitic quarter of a cow is collected prior to milking under sterile conditions as per National Mastitis Council (NMC) guidelines, mixed with an enrichment broth and incubated at 37° C. for about 7 hours. In one embodiment, an aliquot of the enriched milk sample is tested before the next milking (8-12 hours) on a LF test according to the present invention to allow the diagnosis and appropriate antibiotic treatment.

As depicted in FIG. 2, in one embodiment, using a disposable pipette, an aliquot of ≈200-250 μL of the enriched milk sample is added to a test tube. The sample pad portion (2) of the lateral flow dipstick is next immersed in the enriched milk sample in the test tube. A fluid flow then starts which causes the sample to migrate from the sample pad to the adjacent conjugate pad (3).

In one non-limiting embodiment, deposited onto the conjugate pad (3) are different gold conjugates: 1) anti-LTA antibodies that have been conjugated to gold particles and 2) an immunoglobulin which is not specific to Gram positive bacteria, such as chicken IgY, that has been conjugated to gold particles. The gold-conjugated anti-LTA antibodies will form a complex with the gram-positive bacterial LTA antigens present in the milk sample. The formed complex moves along the test strip and forms a complex with anti-LTA antibodies that is striped on the membrane (4) on the test line (5). When the anti-LTA antibody-LTA antigen complex is captured on the sensitized test line (5), its accumulation causes the formation of a clearly visible pink/red band. A pink/red band at the control line ensures that the test is being performed properly. As to the control, in one embodiment, a Chicken IgY gold conjugate on the conjugate pad (3) migrates across the adjacent membrane, where it passes over the test line (4) without reactivity, and crosses another line (control line 6), which has donkey anti-chicken IgY deposited thereon. The Chicken IgY-gold conjugate binds to this control line and the accumulation of the gold colloid particles forms a visible red line.

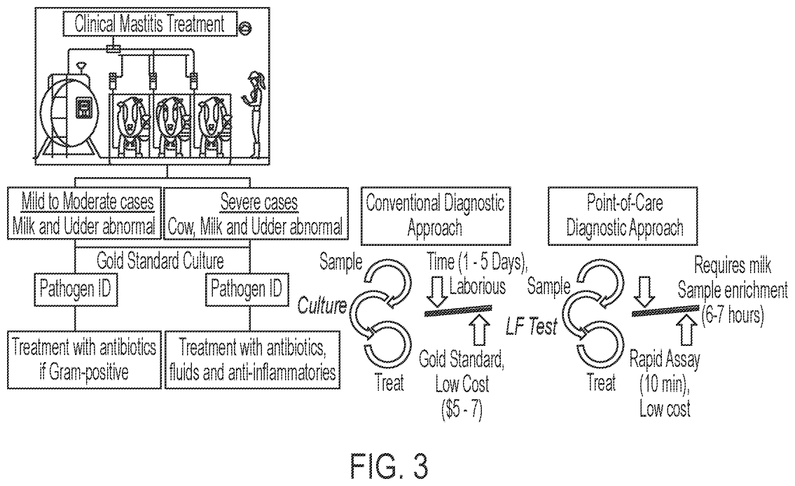

FIG. 3 shows a comparison of a conventional diagnostic approach for detecting mastitis, which employs a gold standard cell culture method for identifying the pathogen versus the point-of care diagnostic approach of the present invention. As shown in the figure, the conventional method can take 1-5 days to identify the pathogen before timely and appropriate antibiotic treatment. In comparison, the point-of-care lateral flow test of the invention employs a milk sample enrichment step which in one embodiment takes about 6-7 hours and which in a preferred embodiment (not depicted) takes about 7-7.5 hours and the test itself can be performed in about 10 minutes. This enables the farmer to obtain results in a timely manner, such as before the next milking. This can help ensure the quality and composition of the milk collected from the farm and reduce financial losses. At the same time, it can improve treatment outcome at least because it guides antimicrobial selection.

The present invention further provides kits comprising one or more of the LF devices described herein and instructions for using the device to detect LTA antigen as an indicator of Gram-positive bacteria in a test sample.

The lateral flow device of the invention detects LTA which is abundantly expressed on the surface of Gram-positive bacteria. In one aspect, the lateral flow device comprises: a) a strip formed of a material enabling capillary flow of an enriched milk sample along a portion of the strip; b) a sample pad located proximal to one end of the strip for receiving the enriched milk sample, c) a conjugate pad located in the strip so that in operation the enriched milk sample flows under capillary action through the strip from the sample pad to the conjugate pad and mobilizes a conjugate contained in the conjugate pad, the conjugate comprising an anti-LTA antibody that has been conjugated to a detection agent, d) a test line comprising an anti-LTA antibody immobilized within the strip along a band located substantially perpendicular to the direction of the sample flow along the strip so that when a formed complex comprising the mobilized anti-LTA antibody conjugate and the LTA in the enriched milk sample contacts the immobilized anti-LTA antibody in the test line, the presence of LTA in the enriched milk sample is indicated by a visible color change; e) a control line located substantially perpendicular to the direction of flow of the enriched milk sample along the strip, the control region being in fluid communication with the sample when it is loaded to the sample loading region; and f) a wicking pad for receiving and retaining the sample after passing through the detection band.

The anti-LTA antibody for use in the present invention can be raised against lipoteichoic acid from a species of Gram-positive bacteria. In one aspect, the anti-LTA antibody is capable of specifically reacting with Gram-positive bacteria lipoteichoic acid in bacterial infected samples. Gram-positive bacteria are characterized by their blue-violet color reaction in the Gram-staining procedure. The color reaction is caused by crystal-violet, the primary Gram-stain dye, complexing with the iodine mordant. When the decolorizer is applied, a slow dehydration of the crystal-violet/iodine complex is observed due to the closing of pores running through the cell wall.

In one embodiment, the anti-LTA antibody is raised against lipoteichoic acid from Staphylococcus epidermidis. In another embodiment, the anti-LTA antibody is raised against lipoteichoic acid from Streptococcus pyogenes. In yet another embodiment, the anti-LTA antibody is raised against lipoteichoic acid from Bacillus subtilis.

Anti-LTA antibodies that have been raised against lipoteichoic acid from Gram-positive bacteria are available commercially. For example, a mouse monoclonal anti-LTA antibody (class IgG1) is commercially available from QED Biosciences, Inc., San Diego, CA (catalog No.: 15711). It was raised against Staphylococcus epidermidis, Hay strain (ATCC #55133) and reacts with lipoteichoic acid of Staphylococcus epidermidis, Hay strain, as well as clinical strains of Staphylococcus epidermidis (types I, II, and III), Staphylococcus aureus strains 5 and 8, Streptococcus pyogenes, Streptococcus fecaelis, and Streptococcus mutans. Also, a mouse monoclonal antibody [clone G43J] to Gram-positive bacteria (ab267414) belonging to class IgG1 is commercially available from Abcam (Cambridge, UK). It is believed that the Abcam antibody was raised against Lipoteichoic acid from Bacillus subtilis. The present inventors have also successfully raised monoclonal antibodies against lipoteichoic acid from Streptococcus pyrogenes using well known methods in the art. Also, a recombinant method was successfully used to clone the antigen-binding region (Fab fragment) of an antibody against lipoteichoic acid from Streptococcus pyrogenes. Lipoteichoic acid from Streptococcus pyogenes can be purchased, for example, from Sigma Aldrich (Cat #L3140-5MG). These are all non-limiting examples of anti-LTA antibodies that can be used in the present invention.

Suitable methods for immobilizing capture entities such as the anti-LTA antibody on solid phases include ionic, hydrophobic, covalent interactions and the like. In terms of immobilizing the conjugates on the conjugate pad, they are typically sprayed onto the conjugate pad with a specialized sprayer similar to an airbrush. The reagent dries on the conjugate pad. Likewise, the test line and control line are striped onto the test strip (e.g., nitrocellulose) with a precision dispensing machine. The proteins bind to the nitrocellulose and are immobilized this way.

The sample pad not only receives milk sample for testing, but removes components from it that might otherwise impede capillary flow of the fluid through the strip or adversely affect detection of the formed LTA antigen-anti-LTA antibody complex at the test line. The milk components that may be removed by the sample pad include cells, cellular material, fats, and particulate matter. For the purpose of detecting LTA antigen in a milk sample from a subject, the sample pad serves as a milk filtering pad that removes milk components, such as cells and fats that might otherwise interfere with the flow of the sample along the strip.

Nitrocellulose was found to be a suitable material from which the strip is made. Other materials may also be suitable provided they allow the desired capillary flow rate and enable suitable detection sensitivity, such as a PVDF membrane, polyethylene membrane, nylon membrane, or a similar type of membrane.

In one embodiment, the milk sample is from an animal, such as but not limited to, a bovine animal. In a specific embodiment, the milk sample is from a dairy cow. In one embodiment, the milk sample is from a mastitic quarter of a cow.

The detection agent is any agent that provides a detectable change when accumulated at the test line or control line. Accumulation of the detection agent at the test line indicates that LTA antigens which are abundantly expressed on the surface of Gram-positive bacteria are present in the milk sample. In practice, the detection agent is conjugated directly or indirectly to an anti-LTA antibody to form a conjugate contained in the conjugate pad that is capable of binding to LTA antigens from the sample. In some embodiments, the conjugate pad can further include a second antibody which is non-specific to Gram-positive bacteria. The second antibody is conjugated directly or indirectly to a detection agent so that in operation, the sample mobilizes the second antibody conjugate which passes over the detection band without reactivity and crosses a control line on which is deposited an antibody capable of binding to the antibody present in the mobilized second antibody conjugate. As the second antibody conjugate crosses the control line and the antibody deposited at the control line binds to it, the binding is indicated by a visible color change.

The accumulation of the detection agent causes a visible color change or observable fluorescence, or any other suitable change at the test line and control line. In various specifically contemplated embodiments of the invention, the detection agent conjugated to the anti-LTA antibody on the conjugate pad is selected from metallic nanoparticles or nanoshells, non-metallic nanoparticles or nanoshells, enzymes, or fluorescent molecules. In specific embodiments, the metallic nanoparticle or metallic nanoshell conjugated to the anti-LTA antibody is selected from gold particles, silver particles, copper particles, platinum particles, cadmium particles, composite particles, gold hollow spheres, gold-coated silica nanoshells, or silica-coated gold shells. In one desired embodiment, the detection agent conjugated to the anti-LTA antibody includes nanoparticles or nanoshells of metallic gold.

In other specifically contemplated embodiments of the invention, the detection agent conjugated to the second antibody (not specific to Gram-positive bacteria) on the conjugate pad is selected from metallic nanoparticles or nanoshells, non-metallic nanoparticles or nanoshells, enzymes, or fluorescent molecules. In specific embodiments, the metallic nanoparticle or metallic nanoshell conjugated to the second antibody is selected from gold particles, silver particles, copper particles, platinum particles, cadmium particles, composite particles, gold hollow spheres, gold-coated silica nanoshells, and silica-coated gold shells.

In one embodiment, it is contemplated that the detection agent conjugated to the anti-LTA anti-body on the conjugate pad may be the same or different from the detection agent conjugated to the second antibody on the conjugate pad. In one desired embodiment, the detection agent conjugated to the anti-LTA antibody and the detection agent conjugated to the second antibody are both gold nanoparticles, to create colloidal gold conjugates.

Methods

The present invention further provides a method for detecting lipoteichoic acid (LTA) as a mastitis Gram-positive bacteria identifier in a milk sample from an animal comprising using a device as described according to any of the embodiments described above. In one desired embodiment, the milk sample has been enriched for bacterial cells. In one embodiment, the milk sample is from a dairy cow. In one specific embodiment, the milk sample is from a mastitic quarter of the cow collected under sterile conditions as per National Mastitis Council (NMC) guidelines. Preferably, the method is performed after the first milking but before the next milking. Typically, cows are milked 2-3 times per day.

The present invention further provides a method for detecting lipoteichoic acid (LTA) as a mastitis gram-positive bacteria identifier. The method includes contacting a milk sample from an animal with a conjugate comprising an anti-LTA antibody that has been conjugated to a detection agent, wherein an antibody-antigen complex is formed between the anti-LTA conjugate and LTA present on the Gram-positive bacteria in the sample; capturing the formed antibody-antigen complex with an anti-LTA antibody; and detecting the captured complex. In a specific embodiment of this method, the milk sample has been enriched for bacterial cells, such as by using the enrichment method and enrichment broth shown in FIG. 2 and described in the example section. Considering the low bacterial load 100 CFU/mL) in many clinical milk samples, the inclusion of a milk sample preparation step wherein the milk sample is first enriched for bacterial cells is desirable. The milk sample enrichment method enables detection of both clinical and sub-clinical cases of mastitis.

In one embodiment of the method, the anti-LTA antibody in the conjugate and used to capture the antibody-antigen complex is a monoclonal antibody.

In a further embodiment of the method, the detection agent conjugated to the anti-LTA antibody is selected from the following: metallic nanoparticles or nanoshells, non-metallic nanoparticles or nanoshells, enzymes, and fluorescent molecules. In a specific embodiment, the detection agent comprises nanoparticles or nanoshells of metallic gold.

The invention is further described with reference to the following examples. It will be appreciated that the invention as claimed is not intended to be limited in any way by these examples.

### EXAMPLES

### Example 1-Milk Sample Enrichment Method

Typically, a lateral flow test alone is insufficient to detect a low bacterial load of ≈100 CFU/mL, which is associated with some mastitic milk samples. In this context, a milk sample preparation method that fits dairy operations and personnel daily activities is desired.

The present example describes the development of an enrichment method. It is based on enriching bacterial cells to a detectable level from a milk sample and subsequent detection on a Gram-positive specific LF test. The milk sample from a mastitic quarter of cow is expected to be collected per NMC guidelines prior to milking, mixed with an enrichment broth and incubated at 37° C. for 7 hours. Finally, an aliquot of enriched milk sample will be tested before the next milking (8-12 hours) on a LF test to allow the diagnosis and appropriate antibiotic treatment.

Five growth media were evaluated: (1) Todd-Hewitt Broth (THB), (2) THB with Colistin and Naladixic Acid (LIM Broth), (3) Brain Heart Infusion (BHI) Broth, (4) Nutrient Broth, and (5) Tryptic Soy Broth (TSB). Milk enrichment methods were tested by incubating different ratios of milk samples and different growth media at 37° C. Following incubation for 7 hours, a mixture of 1.0 mL of milk sample and 1.0 mL of THB supported higher bacterial growth, but also generated undesirable high non-specific binding (NSB) on the LF dipstick. This high NSB was also observed with other four media, but they supported comparatively low bacterial growth. When the beef heart infusion component was removed from the THB, the NSB was eliminated significantly and bacterial growth was demonstrated to be comparable to that observed in THB.

Based on the above findings, a test-specific enrichment broth to mitigate NSB was formulated, which contains Peptone Special from Millipore Sigma (20.0 g/L), Dextrose (2.0 g/L), Sodium chloride (2.0 g/L), Disodium phosphate (0.4 g/L), Sodium Phosphate dibasic (0.4 g/L), Nalidixic acid sodium salt (0.03 g/L) and Sodium carbonate (2.5 g/L). All media components are readily available from commercial sources.

### Example 2—Selection of Critical Reagents

Critical reagents selected for the present assay development preferably detect a wide-range of target Gram-positive bacteria or associated antigens in milk samples. Three different bacterial biomolecules were selected to generate monoclonal antibodies (mAb). Based on their reactivity with target Gram-positive bacteria, commercial anti-lipoteichoic acid (LTA) mAb (QED Bioscience Inc., CA) were selected for LF assay development (Table 1).

TABLE 1
Review of reagents targeted and criteria
for selection of anti-LTA mAbs

| Target | |
|---|---|
| Biomolecules | Initial Outcomes |
| Lipoteichoic | Highly specific to Gram-positive bacteria |
| acid (LTA) | A commercial anti-LTA mAb (QED Bioscience Inc., |
| | San Diego, CA) showed excellent reactivity with target |
| | bacteria and is feasible for LF test development |
| | A recombinant mAb was also generated by cloning the |
| | Fab region of an anti-LTA mAb (QED Biosciences |
| | Inc., San Diego, CA) as a back-up option and is |
| | feasible for LF test development. |
| Peptidoglycan | Anti-PGN mAbs specific to S. aureus were generated |
| (PGN) | in-house |
| | Anti-PGN mAbs failed to detect bacterial cells in |
| | ELISA-based screening |
| Surface proteins | rSdrD and rScaB-specific mAbs were generated in- |
| | house, which failed to detect whole bacterial cells in |
| | ELISA-based screening |

A monoclonal anti-LTA antibody (class IgG1) commercially available from QED Biosciences, Inc., San Diego, CA (catalog No.: 15711) was selected for LF test development. This antibody was raised against: Staphylococcus epidermidis, Hay strain (ATCC #55133). The host Species was mouse. The anti-LTA antibody reacts with lipoteichoic acid of Staphylococcus epidermidis, Hay strain, as well as clinical strains of Staphylococcus epidermidis (types I, II, and III), Staphylococcus aureus strains 5 and 8, Streptococcus pyogenes, Streptococcus fecaelis, and Streptococcus mutans. It does not react with peptido-glycan of Staphylococcus aureus or peptidoglycan-rhamnose, nor does it react with pneumococcal polysaccharides. This antibody does not cross-react with E. coli or H. influenzae type B.

### Example 3 Development of a Preliminary Lateral Flow Dipstick

The present example describes one embodiment of the device and method of the present invention. A lateral flow-based, sandwich immunoassay format was selected. The test consists of a nitrocellulose membrane laminated to an adhesive backing card. Both ends of the nitrocellulose membrane are overlapped by an adjacent conjugate pad and an adjacent absorption pad. A sample pad overlaps the conjugate pad. After deposition of all reagents to the respective membranes, the card is cut into strips approximately 5 mm-wide.

The test strip architecture consisted of the components disclosed in Table 2. Immunoassays were performed on clinical milk samples that had been enriched for bacterial cells according to the method and preferred enrichment media described in Example 1, using the test protocol described in Example 4.

TABLE 2
Lateral flow dipstick using anti-LTA mAb (QED Bioscience Inc., CA)
FORMAT

| | |
|---|---|
| Test Strip | Nitrocellulose membrane (MDI Membrane |
| | Technologies, India) |
| Test Line | 1.25 mg/mL of anti-LTA mAb (QED Bioscience |
| | Inc.); diluted with test line solution (phosphate |
| | buffer with stabilizing sugars) and sprayed at the |
| | test line |
| Control Line | 1.0 mg/mL of donkey anti-chicken IgY antibody |
| | (Jackson Immunoresearch); diluted in test line |
| | solution (phosphate buffer with stabilizing sugars) |
| | and sprayed at the control line |
| Absorbent pad | 100% cotton chromatography paper (Ahlstrom- |
| | Munksjö, Helsinki, Finland) |
| Gold Conjugate & | chopped glass w/binder conjugate pad (Ahlstrom- |
| Conjugate pad | Munksjö, Helsinki, Finland), sprayed with (a) anti- |
| | LTA mAb (QED) and nanoComposix gold |
| | conjugate, and (b) chicken IgY and nanoComposix |
| | gold conjugate diluted in the conjugate solution |
| Sample pad | CytoSep ® single layer media consisting of high |
| | purity natural & synthetic fibers (Ahlstrom- |
| | Munksjö, Helsinki, Finland) |

The conjugation can be prepared using standard antibody conjugation methods to colloidal gold. The anti-LTA antibody is mixed with a buffer at a desired pH. The colloidal gold (nanoComposix, San Diego, CA) is added to the antibody and mixed for 5-10 minutes. A second basic buffer is added to the conjugate to raise the pH, and the conjugate is blocked by the addition of BSA.

To prepare the control conjugate, colloidal gold is adjusted to a desired pH. A saturating quantity of protein (e.g., chicken IgY) between 20 and 100 μg/ml is added to the gold and incubated for 10 minutes. A BSA blocker is then added to the gold and incubated for an additional 10 minutes. A stabilizer buffer including BSA and sucrose is added to the conjugate.

The conjugates are mixed together at a critical, optimized OD with a conjugate diluent consisting of detergents, buffer, sucrose, and BSA. The conjugates are sprayed onto the conjugate pad with an airjet sprayer.

The test and control line reagents, anti-LTA antibody, and donkey anti-chicken IgY, respectively, are diluted to an optimized, critical concentration in a deposition buffer with stabilizing sugars. The reagents are deposited onto the nitrocellulose with a high precision fluidic handler, capable of spraying micro quantities of volume. The cards are stored at <30% relative humidity.

### Example 4-Test Protocol

Add 1.0 mL of milk sample to a graduated tube containing 1.0 mL of enrichment broth
Incubate the tubes with milk and enrichment broth mix at 37° C. for 7 hours in a routine bacteriological incubator (no specialized milk sample prep device is required).
Following incubation, test the enriched milk sample (200-250 μL) by placing a LF dipstick into the enriched milk sample.
LTA antigen abundantly expressed on the surface of Gram-positive bacteria in the enriched milk sample will migrate to the conjugate pad and react with anti-LTA antibody conjugated to colloidal gold. A complex is formed between the anti-LTA antibody conjugate and LTA in the sample. The formed complex migrates across the nitrocellulose where the complexed LTA is immobilized by anti-LTA antibody deposited on the test line. The accumulation of colloidal gold particles on the test line form a visible red line if LTA antigen is present, indicating a positive result. If LTA antigen is not present in the sample, the gold conjugate is not immobilized on the test line and continues to migrate to the absorbent pad. The lack of formation of a red line on the test line indicates the sample is negative for LTA, which means that Gram-positive bacteria are not present. In one embodiment, a second conjugate deposited onto the conjugate pad—a control conjugate—consists of chicken IgY conjugated to colloidal gold. The control conjugate migrates across the nitrocellulose and is immobilized on a second reaction line—the control line—by an anti-chicken IgY antibody. The accumulation of colloidal gold control conjugate particles forms a red control line. The control line is a procedural control and indicates the test was performed correctly and flowed correctly.
The test strips are read visually after 10 minutes.

### Example 5—Sample Collection

The sample set consisted of the following, which were received overnight on ice from different dairies:
Clinical milk samples (n=108) with 100 CFU/mL of target Gram-positive bacteria derived from infected quarters of cows
Clean and culture-negative milk samples (n=103) derived from mastitis-free quarters of COWS

Etiological mastitis pathogen(s) present in each clinical milk sample were enumerated and identified by direct culture, followed by MALDI-ToF analysis.

### Example 6—Test Results-Limit of Detection (LoD) of Enrichment-Based Lateral Low Assay

The Limit of detection (LoD) of the alpha prototype test was estimated using milk samples spiked with different concentrations (CFU/mL) of each target Gram-positive bacterium. Each enriched sample was tested in 10 replications and the concentration (CFU/mL) at which the assay is visually positive in 9 out of 10 replications was considered as LoD specific to each target bacterium.

TABLE 3
Estimation of LoD of enrichment-based lateral flow assay

| Target Gram-positive Pathogen | LoD (CFU/mL) |
|---|---|
| Staph. aureus | 63 |
| Staph. chromogenes (CNS) | 43 |
| Staph. epidermidis (CNS) | 30 |
| Staph. haemolyticus (CNS) | 40 |
| Staph. hyicus (CNS) | 57 |
| Staph. simulans (CNS) | 5 |
| Staph. xylosus (CNS) | 25 |
| Strep. uberis | 32 |
| Strep. agalactiae | 13 |
| Strep. dysgalactiae | 20 |

### Example 7-Estimation of Preliminary Diacinostic Performance

Following enrichment for 7 hours at 37° C., an aliquot of each enriched milk sample was tested on three different lots of LF dipsticks. After 10 minutes, visual results were recorded. Using culture results as the reference:
Test strip lots #1 and 2 detected target Gram-positive bacteria in 105/108 clinical milk samples. Estimates of diagnostic sensitivity and specificity were 97.2% (95% CI: 92.1-99.4%) and 95.1% (95% CI: 89.0-98.4%), respectively (Table 4).
   Test strip lot #3 produced 104/108 positive results. Estimates of diagnostic sensitivity and specificity were 96.3% (95% CI: 90.8-99.0%) and 96.1% (95% CI: 90.4-98.9%), respectively (Table 4).
Three false negative and five false positive results were observed with Lot #1 and 2; whereas, four false negative and four false positive results were observed with Lot #3.

TABLE 4
Estimates of sensitivity and specificity of enrichment-based lateral flow assay

| | | | | | | Sensitivity | Specificity |
|---|---|---|---|---|---|---|---|
| | | | | | Total | (95% | (95% |
| Test | True | False | True | False | Number | Confidence | Confidence |
| Strip | Positive | Negative | Negative | Positive | of Tests | Interval) | Interval) |
| Lot # | 105 | 3 | 98 | 5 | 211 | 97.2% | 95.1% |
| 1 | | | | | | (92.1-99.4%) | (89.0-98.4%) |
| Lot # | 105 | 3 | 98 | 5 | 211 | 97.2% | 95.1% |
| 2 | | | | | | (92.1-99.4%) | (89.0-98.4%) |
| Lot # | 104 | 4 | 99 | 4 | 211 | 96.3% | 96.1% |
| 3 | | | | | | (90.8-99.0%) | (90.4-98.9%) |

### Example 8—Instrumentation

In one embodiment, the result interpretation for this LF test is based on visual assessment by a human technician. It is designed for simple set-up, limited hands-on time, and ease of read-out. This kit does not require a sophisticated lateral flow reader and associated software, although the LF test can be based on assessment of test results by a lateral flow reader, if desired. However, a simple bacteriological incubator (e.g., a heat block) with the ability to maintain a temperature of 37° C. is required for the milk sample enrichment step. Dairies that practice on-farm culturing have these incubators in the dairy office or dairy lab. In one embodiment, portable incubators can be provided for those dairies that do not have an incubator.

1. A lateral flow device for detecting lipoteichoic acid (LTA) expressed on the surface of Gram-positive bacteria as a mastitis Gram-positive bacteria identifier in a milk sample of an animal, the device comprising: a) a strip formed of a material enabling capillary flow of fluid along a portion of the strip; b) a sample pad located proximal to one end of the strip for receiving the milk sample, c) a conjugate pad located in the strip so that in operation the sample flows under capillary action through the strip from the sample pad to the conjugate pad and mobilizes a conjugate contained in the conjugate pad, the conjugate comprising an anti-LTA antibody that has been conjugated to a detection agent, d) a test line comprising an anti-LTA antibody immobilized within the strip along a band located substantially perpendicular to the direction of sample flow along the strip so that when a formed complex comprising the mobilized anti-LTA antibody conjugate and LTA in the sample contacts the immobilized anti-LTA antibody in the test line the presence of LTA in the sample is indicated by a visible color change.

2. The device of claim 1, wherein the milk sample has been enriched for bacterial cells.

3. The device of claim 1, wherein the milk sample is from a mastitic quarter of a cow.

4. The device of claim 1, further comprising a wicking pad for receiving and retaining sample after passing through the test line.

5. The device of claim 1, wherein the anti-LTA antibody in the conjugate and in the test line is a monoclonal antibody.

6. The device of claim 1, wherein the detection agent conjugated to the anti-LTA antibody is selected from the group comprising metallic nanoparticles or nanoshells, non-metallic nanoparticles or nanoshells, enzymes, and fluorescent molecules.

7. The device of claim 6, wherein the detection agent comprises nanoparticles or nanoshells of metallic gold.

8. The device of claim 1, wherein the lateral flow device is a dipstick.

9. The device of claim 8, wherein the sample pad portion of the dipstick is immersible in the milk sample.

10. The device of claim 1, wherein the strip is housed within a cassette.

11. The device of claim 1, wherein the sample pad comprises a filter membrane for removing one or more components from the sample.

12. The device of claim 11, wherein the one or more components removed from the sample by the filter membrane of the sample pad are cells, cellular material, fats, or particulate matter.

13. The device of claim 1, further comprising a control line located substantially perpendicular to the direction of flow of the sample along the strip.

14. The device of claim 13, wherein the conjugate pad further comprises an antibody not specific to Gram-positive bacteria, which is conjugated to a detection agent to form a second antibody conjugate so that in operation the sample flows from the sample pad to the conjugate pad and mobilizes the second antibody conjugate which passes over the test line without reactivity and crosses the control line.

15. The device of claim 14, wherein deposited at the control line is an antibody capable of binding to the mobilized second antibody conjugate as it crosses the control line, said binding at the control line being indicated by a visible color change.

16. The device of claim 14, wherein the antibody in the second antibody conjugate is from animal species other than the species from which the milk sample is derived.

17. The device of claim 14, wherein the detection agent conjugated to the antibody in the second antibody conjugate is selected from the group comprising metallic nanoparticles or nanoshells, non-metallic nanoparticles or nanoshells, enzymes, and fluorescent molecules.

18. The device of claim 17, wherein the detection agent conjugated to the antibody in the second antibody conjugate comprises nanoparticles or nanoshells of metallic gold.

19. The device of claim 1, wherein the strip is formed of nitrocellulose.

20. The device of claim 1, wherein the milk sample is from an animal selected from the group consisting of a canine, a feline, equine, caprine, ovine, or a bovine animal.

21. The device of claim 1, wherein the milk sample is from a bovine animal.

22. A method for detecting lipoteichoic acid (LTA) as a mastitis gram-positive bacteria identifier in a milk sample from an animal comprising using a device of any one of claim 1.

23. A method for detecting lipoteichoic acid (LTA) as a mastitis gram-positive bacteria identifier, the method comprising contacting a milk sample from an animal with a conjugate comprising anti-LTA antibody that has been conjugated to a detection agent, wherein an antibody-antigen complex is formed between the anti-LTA conjugate and LTA present on the Gram-positive bacteria in the sample; capturing the formed antibody-antigen complex with an anti-LTA antibody; and detecting the captured complex.

24. The method of claim 23, wherein the milk sample has been enriched for bacterial cells.

25. The method of claim 23, wherein the anti-LTA antibody in the conjugate and used to capture the antibody-antigen complex is a monoclonal antibody.

26. The method of claim 23, wherein the detection agent conjugated to the anti-LTA antibody is selected from the group comprising metallic nanoparticles or nanoshells, non-metallic nanoparticles or nanoshells, enzymes, and fluorescent molecules.

27. The method of claim 23, wherein the detection agent comprises nanoparticles or nanoshells of metallic gold.
